(19) United States
(12) Patent Application Publication
(10) Pub. No.: US 2026/0187791 A1
(43) Pub. Date: Jul. 2, 2026

(54) SINGLE ML/AI MODEL ON MULTIPLE ULTRASOUND PLATFORMS AND FOR MULTIPLE SCANNING MODES

(71) Applicant: GE Precision Healthcare LLC, Waukesha, WI (US)

(72) Inventors: Michael G. Cannon, Haverford, PA (US); Nicolas Poilvert, Denver, CO (US); Matthew Howard Self, Emerald Hills, CA (US); Fernand Dominique Pajot, Boulder, CO (US)

(21) Appl. No.: 19/008,164

(22) Filed: Jan. 2, 2025

(51) Int. Cl. G06T 7/00; A61B 8/00; A61B 8/06; G16H 30/40

(52) U.S. Cl. CPC G06T 7/0012; A61B 8/06; A61B 8/488; A61B 8/5246; G16H 30/40; G06T 2207/10132; G06T 2207/20081; G06T 2207/30104; G06T 2207/30168

(57) ABSTRACT

Disclosed herein are ultrasound imaging systems which provide a dual-mode guided imaging procedure using machine learning systems, which can be used, for example, to assist in acquisition of diagnostic images for assessing a health condition of a target organ of a subject.

### BACKGROUND

Ultrasound imaging is a non-invasive diagnostic modality that is portable and affordable. It does not use ionizing radiation and can be applied in a wide range of medical applications. Certain imaging systems process the ultrasound images prior to submission to machine learning (ML) systems. In some instances, this provides a reduced image quality. In some instances, the dual-mode ultrasound imaging system and process described herein improves image quality by submitting unprocessed images to a ML system described herein. In some instances, the dual-mode ultrasound imaging system and process described herein involves imaging tissue and blood flow to provide exhaustive clinical measurements and involves one or more modes of interest such as B-mode, Color Doppler mode, Spectral Doppler mode, and zoom mode. Each mode requires different combinations of imaging settings, such as resolution, frame rates, or contrast to produce optimal image quality. Accordingly, described herein are methods and systems that submit unprocessed images to the machine learning system to determine the user metrics, which provides overall higher image quality.

### SUMMARY

In some aspects, described herein, is an ultrasound imaging system configured for conducting a dual-mode guided ultrasound imaging procedure, the system comprising: an ultrasound imaging probe; a computing system; and a computer-readable storage medium, storing instructions that, when executed by a processor of the computing system cause the ultrasound imaging system to: a first plurality of images comprising a first mode and a second mode, the first mode comprising a B-mode ultrasound imaging mode, and the second mode comprising a zoom ultrasound imaging mode; an unprocessed first mode image of the first plurality of images to a machine learning model to determine one or more user metrics comprising a contemporaneous image quality score and/or a guided movement expected to improve an image quality of a subsequently acquired second plurality of images compared to an image quality of the first plurality of images, based at least in part on the unprocessed first mode image of the first plurality of images; and wherein the one or more user metrics are provided to a user of the ultrasound imaging system.

In some aspects, described herein, is a method for conducting a dual-mode guided ultrasound imaging produce, the method comprising: obtaining a first plurality of images from an ultrasound imaging system, the first plurality of images comprising a first mode and a second mode, the first mode comprising a B-mode ultrasound imaging mode, and the second mode comprising a zoom ultrasound imaging mode; submitting an unprocessed first mode image of the first plurality of images to a machine learning model to determine one or more user metrics comprising a contemporaneous image quality score and/or a guided movement expected to improve an image quality of a subsequently acquired second plurality of images compared to an image quality of the first plurality of images, based at least in part on the unprocessed first mode image of the first plurality of images; and providing the one or more user metrics to a user of the ultrasound imaging system.

In some aspects, the first plurality of images comprises multiple, repetitive images.

In some aspects, the B-mode depicts an anatomical structure.

In some aspects, the zoom ultrasound imaging mode magnifies the target feature in the second plurality of images together with a user readable structural image of the first mode.

In some aspects, the zoom ultrasound imaging mode magnifies the target feature in a target organ.

In some aspects, the zoom ultrasound imaging mode magnifies one or more aspects including depth, angle, and line density.

In some aspects, the improvement in image quality improves the raw image quality.

In some aspects, the raw image quality is improved by one or more aspects including reducing pixelation, noise, clutter, or speckle, or increased clarity or contrast.

In some aspects, the improvement in image quality comprises an improvement in visualization of a target structural feature of a target organ of a subject.

In some aspects, further comprising: (d) the first mode image of the second plurality of ultrasound images; (e) the unprocessed first mode image of (d) is submitted to a machine learning model to obtain one or more updated user metrics instructions; and (f) the updated user metrics instructions are provided to the user.

In some aspects, further comprising repeating (d) and (e) in real-time.

In some aspects, the user metrics are provided until a target view is reached.

In some aspects, the target view is determined based at least in part on the diagnostic procedure.

In some aspects, the user metrics are provided until a threshold quality is reached.

In some aspects, the threshold quality depends upon a presence of a target structural feature of an organ, a clarity of structural image quality, Doppler image quality, or a combination thereof.

In some aspects, the unprocessed second mode data is used to determine the user metrics.

In some aspects, the first mode is used to determine one or more aspects including current, threshold quality, or target view; and wherein the second mode is used to determine one or more aspects including current, threshold quality, or target view.

In some aspects, the unprocessed first mode image comprises a plurality of raw images that have not been enhanced for human-readability and are not in a displayable format; and wherein the unprocessed second mode image comprises a plurality of raw images that have not been enhanced for human-readability and are not in a displayable format.

In some aspects, disclosed herein, is a computer system for conducting a dual-mode guided ultrasound imaging procedure, the computer system configured to: receive a first plurality of images comprising a first mode and a second mode, the first mode comprising a B-mode ultrasound imaging mode, and the second mode comprising a zoom ultrasound imaging mode; submit an unprocessed first mode image of the first plurality of images to a machine learning model to determine one or more user metrics comprising a contemporaneous image quality score and/or a guided movement expected to improve an image quality of a subsequently acquired second plurality of images compared to an image quality of the first plurality of images, based at least in part on the unprocessed first mode image of the first plurality of images; and provide the one or more user metrics to a user of the ultrasound imaging system.

In some aspects, disclosed herein, is an ultrasound imaging system configured for conducting a dual-mode guided ultrasound imaging procedure, the system comprising: an ultrasound imaging probe; a computing system; and a non-transitory computer-readable storage medium, storing instructions that, when executed by a processor of the computing system cause the ultrasound imaging system to: obtain a first plurality of images comprising a first mode and a second mode, the first mode comprising a B-mode ultrasound imaging mode, and the second mode comprising a Doppler flow ultrasound imaging mode; submit an unprocessed first mode image of the first plurality of images to a machine learning model to determine one or more user metrics comprising a contemporaneous image quality score and/or a guided movement expected to improve an image quality of a subsequently acquired second plurality of images compared to an image quality of the first plurality of images, based at least in part on the unprocessed first mode image of the first plurality of images; and provided the one or more user metrics to a user of the ultrasound imaging system.

In some aspects, disclosed herein, is a method for conducting a dual-mode guided ultrasound imaging produce, the method comprising: obtaining a first plurality of images from an ultrasound imaging system, the first plurality of images comprising a first mode and a second mode, the first mode comprising a B-mode ultrasound imaging mode, and the second mode comprising a Doppler flow ultrasound imaging mode; submitting an unprocessed first mode image of the first plurality of images to a machine learning model to determine one or more user metrics comprising a contemporaneous image quality score and/or a guided movement expected to improve an image quality of a subsequently acquired second plurality of images compared to an image quality of the first plurality of images, based at least in part on the unprocessed first mode image of the first plurality of images; and providing the one or more user metrics are provided to a user of the ultrasound imaging system.

In some aspects, the first plurality of images comprises multiple, repetitive images.

In some aspects, the dual-mode guided ultrasound imaging procedure identifies a target organ.

In some aspects, the B-mode depicts an anatomical structure.

In some aspects, the Doppler flow ultrasound imaging mode comprises providing a color map of blood flow together with a user readable structural image of the first mode.

In some aspects, the Doppler flow ultrasound imaging mode comprises providing a spectral histogram of blood flow together with the user readable structural image of the first mode.

In some aspects, the improvement in image quality improves the raw image quality.

In some aspects, the improvement in raw image quality comprises a reduction in pixelation, noise, clutter, or speckle, or increased clarity or contrast.

In some aspects, the improvement in image quality comprises an improvement in visualization of a target structural feature of the target organ of a subject.

In some aspects, further comprising: (d) the first mode image of the second plurality of images; (e) the unprocessed first mode image of (d) is submitted to a machine learning model to obtain one or more updated user metrics; and (f) the updated user metrics are provided to the user.

In some aspects, further comprising repeating (d) and (e) in real-time.

In some aspects, the user metrics are provided until a target view is reached.

In some aspects, the target view is determined based at least in part on the diagnostic procedure.

In some aspects, the user metrics are provided until a threshold quality is reached.

In some aspects, the threshold quality depends upon a presence of a target structural feature of an organ, a clarity of structural image quality, Doppler image quality, or a combination thereof.

In some aspects, wherein blood flow takes place in the target organ.

In some aspects, an unprocessed second mode data is used to determine the user metrics.

In some aspects, wherein the first mode is used to determine one or more aspects including current, threshold quality, or target view; and wherein the second mode is used to determine one or more aspects including current, threshold quality, or target view.

In some aspects, wherein the unprocessed first mode image comprises a plurality of raw images that have not been enhanced for human-readability and are not in a displayable format; and wherein the unprocessed second mode image comprises a plurality of raw images that have not been enhanced for human-readability and are not in a displayable format.

In some aspects, disclosed herein, is a computer system for conducting a dual-mode guided ultrasound imaging procedure, the computer system configured to: receive a first plurality of images comprising a first mode and a second mode, the first mode comprising a B-mode ultrasound imaging mode, and the second mode comprising a Doppler flow ultrasound imaging mode; submit an unprocessed first mode image of the first plurality of images to a machine learning model to determine one or more user metrics comprising a contemporaneous image quality score and/or a guided movement expected to improve an image quality of a subsequently acquired second plurality of images compared to an image quality of the first plurality of images, based at least in part on the unprocessed first mode image of the first plurality of images; and provide the one or more user metrics to a user of the ultrasound imaging system.

Additional aspects and advantages of the present disclosure will become readily apparent to those skilled in this art from the following detailed description, wherein only illustrative aspects of the present disclosure are shown and described. As will be realized, the present disclosure is capable of other and different aspects, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative aspects, in which the principles of the invention are utilized, and the accompanying drawings (also “Figure” and “FIG.” herein), of which:

FIG. 1 illustrates an example workflow for guiding an ultrasound imaging procedure according to aspects described herein.

FIG. 2 illustrates another example workflow for guiding an ultrasound imaging procedure according to aspects described herein.

FIG. 3 illustrates an example workflow for guiding an ultrasound imaging procedure according to aspects described herein.

FIG. 4 illustrates another workflow for guiding an ultrasound imaging procedure according to aspects described herein.

FIG. 5 illustrates an example workflow for guiding an ultrasound imaging procedure according to aspects described herein.

FIG. 6 illustrates another example workflow for processing images using either first mode or second mode according to aspects described herein.

FIG. 7 illustrates an example workflow for processing images using first mode and second mode according to aspects described herein.

FIG. 8 illustrates example workflow for processing images using first mode and second mode according to aspects described herein.

FIG. 9 illustrates another example workflow for processing images using first mode and second mode according to aspects described herein.

FIG. 10 illustrates an example workflow processing images using B-mode and Zoom mode according to aspects described herein.

FIG. 11 illustrates an example workflow processing images using B-mode and Color mode according to aspects described herein.

FIG. 12 shows a computer system that is programmed or otherwise configured to implement methods provided herein.

FIGS. 13A-B illustrate processed images of the blood-filled chambers in human cardiac structures.

FIGS. 14A-D illustrates Color mode imaging of various processes in the human body. FIG. 14A illustrates Color mode imaging of the umbilical artery. FIG. 14B illustrates Color mode imaging of the kidney blood flow. FIG. 14C illustrates Color mode imaging of the carotid artery. FIG. 14D illustrates Color mode imaging of the aortic valve regurgitation.

FIGS. 15A-B illustrates an overlay of Spectral Doppler mode, Color mode, and B-mode imaging of various processes in the human body. FIG. 15A illustrates the overlay of Spectral Doppler mode, Color mode, and B-mode imaging of the heart. FIG. 15B illustrates the overlay of Spectral Doppler mode, Color mode, and B-mode imaging of the carotid artery.

FIGS. 16A-B illustrates an example of mitral valve regurgitation. FIG. 16A illustrates an example of mitral valve regurgitation with high image quality. FIG. 16B illustrates an example of mitral valve regurgitation with poor image quality.

### DETAILED DESCRIPTION

While various aspects of the invention have been shown and described herein, it will be obvious to those skilled in the art that such aspects are provided by way of example only. Numerous variations, changes, and substitutions may occur to those skilled in the art without departing from the invention. It should be understood that various alternatives to the aspects of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive aspects herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

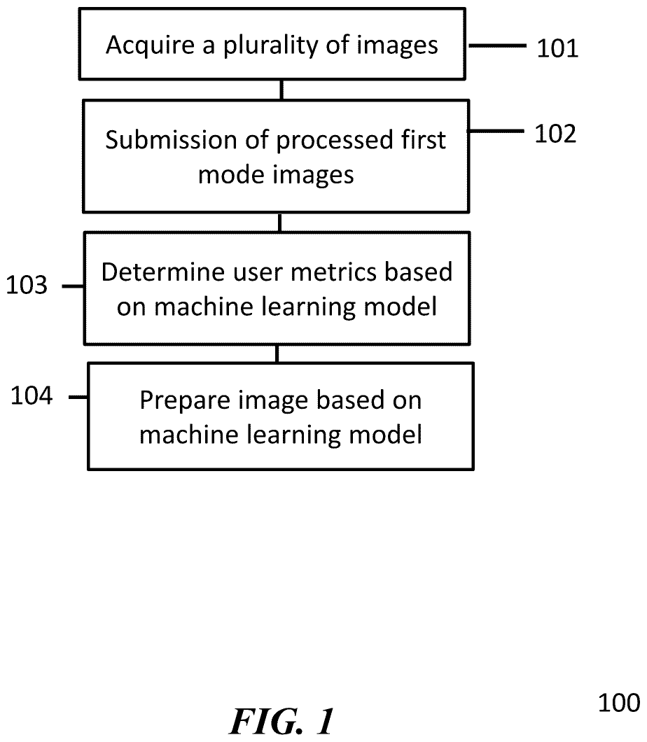

Methods and systems described herein can be implemented according to numerous alternative workflows. For example, the workflow 100 illustrated in FIG. 1 involves: acquiring a plurality of images from a subject 101, for example, using an imaging probe of an ultrasound imaging system (e.g., using a transducer of a two-dimensional ultrasound imaging system such as a linear probe, a curvilinear probe, or a phased array probe). In some embodiments, the patient or subject is an individual seeking treatment. In some embodiments, the patient or subject has a disease or disorder. In some embodiments, the patient or subject is considered healthy and/or performing a routine health check. In some embodiments, the patient or subject is a human. The workflow described herein involves submitting the processed first mode images 102, wherein the first mode images are used to determine the user metrics based on a machine learning (ML) model 103. The first mode may comprise B-mode processing. B-mode processing may be used to capture the anatomical structures in the subject. A processed image may be edited for human readability (e.g., by amending, augmenting, or annotating a raw image by any of the annotations described herein); whereas a raw image may be an acquired image prior to post-acquisition image enhancements (e.g., using filters to improve raw image quality or annotations to improve human readability). User metrics provided herein may be used to assist a user in acquiring subsequent ultrasound images having an improved raw image quality (e.g., reducing pixelation, clutter, speckle, or noise; and improving clarity and contrast) and/or an improved clinical or diagnostic quality as described herein. In some cases the user metrics comprise providing guided movement instructions to a user. In some cases, the user metrics comprise providing one or more indicators of image quality to a user (e.g., raw image quality, clinical or diagnostic quality, and/or combinations thereof). In some instances, the one or more indicators of image quality comprises one or more quantitative quality metrics. The ML model may be used to determine a contemporaneous image quality score and/or a guided movement. The contemporaneous image quality score may be used to further direct a guided movement. The guided movement may be used to improve the image quality. The image may be prepared using the ML model 104.

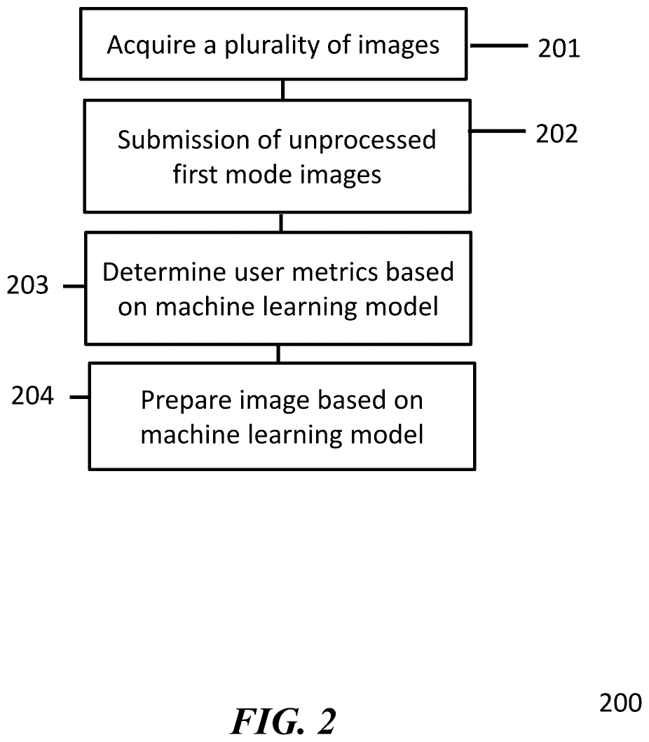

FIG. 2 outlines the workflow described herein 200. The workflow involves: acquiring a plurality of images from a subject 201, for example, using an imaging probe of an ultrasound imaging system (e.g., using a transducer of a two-dimensional ultrasound imaging system such as a linear probe, a curvilinear probe, or a phased array probe). The unprocessed image may be submitted to a ML system 202. The ML system may provide user metrics. The user metrics may, for example, determine a contemporaneous image quality score and/or a guided movement 203. The image may be prepared using the ML model 204. The guided movement can be directed to enhance the raw image quality (e.g., reducing pixelation, speckle, clutter, or noise; and improving clarity and contrast). The workflow described herein provides improved image quality.

In some embodiments, the ideal image quality may vary depending on clinical or diagnostic quality. In some embodiments, the clinical or diagnostic quality depends on the user's ability to perform a diagnosis. Diagnosis factors may include clarity, presence, and/or structural visibility of target view and/or target structure.

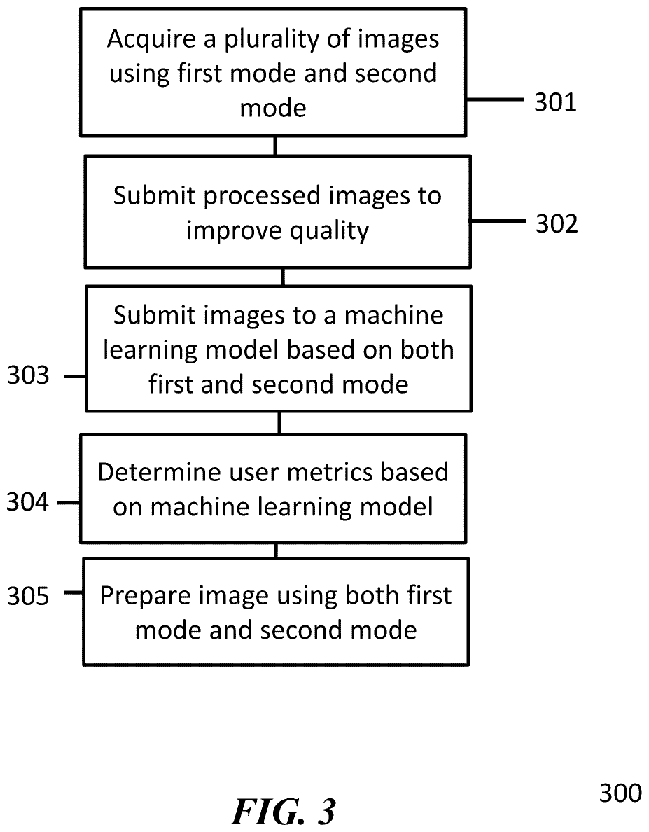

The workflow 300 illustrated in FIG. 3 comprises: acquiring a plurality of images using first mode and second mode 301. The images may be captured, for example, using an imaging probe of an ultrasound imaging system (e.g., using a transducer of a two-dimensional ultrasound imaging system such as a linear probe, a curvilinear probe, or a phased array probe). The images may be processed 302. The processed images may, for example, be submitted to a ML model based on both first mode and second mode 303. The first mode may comprise B-mode processing. B-mode processing may comprise capturing anatomical structures. The second mode may comprise Zoom mode. Zoom mode may depict the high depth details of the anatomical structures. In some embodiments, the second mode may comprise Color Doppler mode. Color Doppler mode may comprise depicting the movement of blood flow in anatomical structures. The ML model may determine user metrics to improve the image quality 304. The user metrics may provide a contemporaneous image quality score and/or a guided movement. The contemporaneous image quality score may provide a metric to measure the quality of the plurality of images. The contemporaneous image quality score may be used to direct the guided movements. The guided movements may be used to improve the image quality (e.g., improve contrast and clarity; and reduce pixelation, noise, clutter, or speckle) 305.

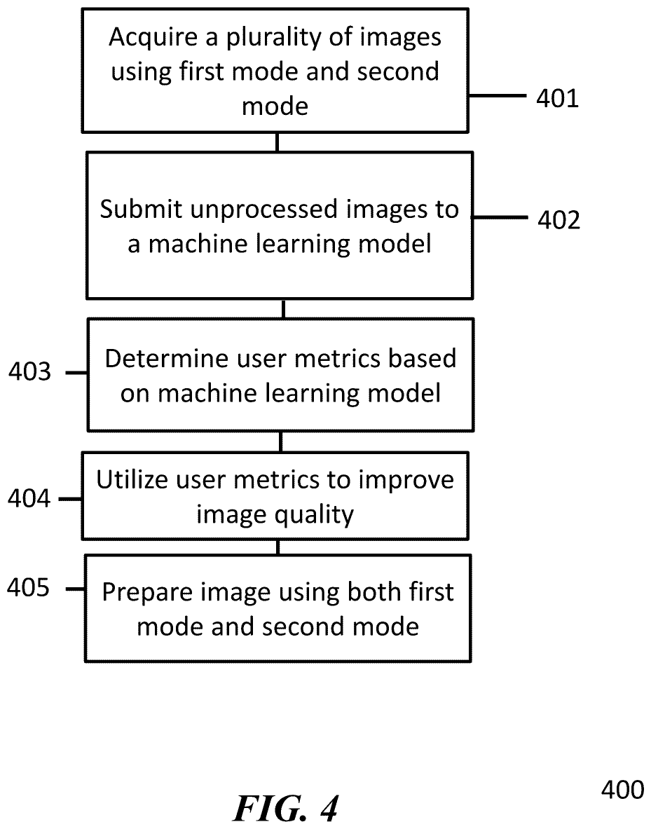

The workflow 400 as described herein is illustrated in FIG. 4. FIG. 4 involves: acquiring a plurality of images using first mode and second mode 401. The first mode may comprise B-mode processing. B-mode processing may comprise capturing anatomical structures. The second mode may comprise Zoom mode. Zoom mode may depict the high depth details of the anatomical structures. In some embodiments, the second mode is Color Doppler mode. Color Doppler mode may comprise depicting the movement of blood flow in anatomical structures. The images can be captured, for example, using an imaging probe of an ultrasound imaging system (e.g., using a transducer of a two-dimensional ultrasound imaging system such as a linear probe, a curvilinear probe, or a phased array probe). The images may be unprocessed 402, so that they are not altered for human readability. The unprocessed images may be submitted to a ML model. The ML model may be used to determine user metrics 403. The user metrics may provide a contemporaneous image quality score and/or a guided movement. The ML model may be used to annotate and distinguish features. The ML model may utilize the user metrics to improve the image quality 404. The user metrics may direct the guided movement to enhance the image quality (e.g., reducing pixelation, speckle, clutter, or noise; and improving clarity and contrast). The user metrics may be used to prepare the images using both the first mode and second mode 405.

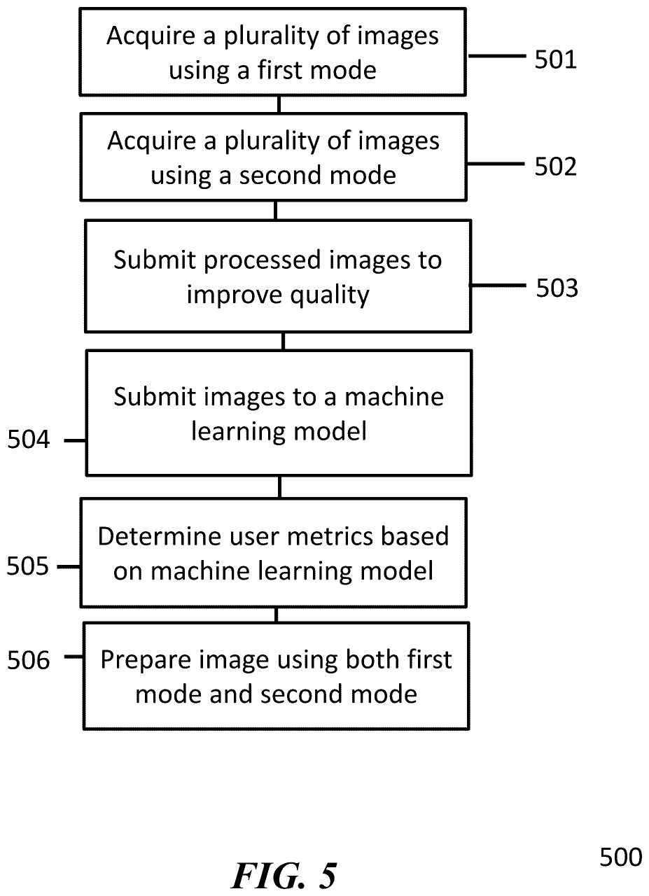

The workflow as described herein 500 is illustrated in FIG. 5. FIG. 5 comprises: acquiring a plurality of first mode images and second mode images. The first mode images may use B-mode to capture anatomical structures 501. The second mode images may acquire a plurality of images using Color Doppler mode to capture blow flow movement in the anatomical structures. The second mode can use Zoom mode to capture high depth details of the anatomical structures 502. The plurality of images captured by first mode or second mode may involve, for example, using an imaging probe of an ultrasound imaging system (e.g., using a transducer of a two-dimensional ultrasound imaging system such as a linear probe, a curvilinear probe, or a phased array probe). The plurality of images may be processed 503. The processed images may further, for example, be submitted to a ML model as described herein 504. The processed images may have been altered previously for human readability. The processed images may be used to determine user metrics 505. The user metrics may provide a contemporaneous image quality score and/or a guided movement. The guided movements may direct enhancements to the image quality (e.g., reducing pixelation, speckle, clutter, or noise; and improving clarity and contrast) The user metrics may be continually updated according to new processing data. The user metrics associated with both the first mode and second mode will be used to prepare the image 506.

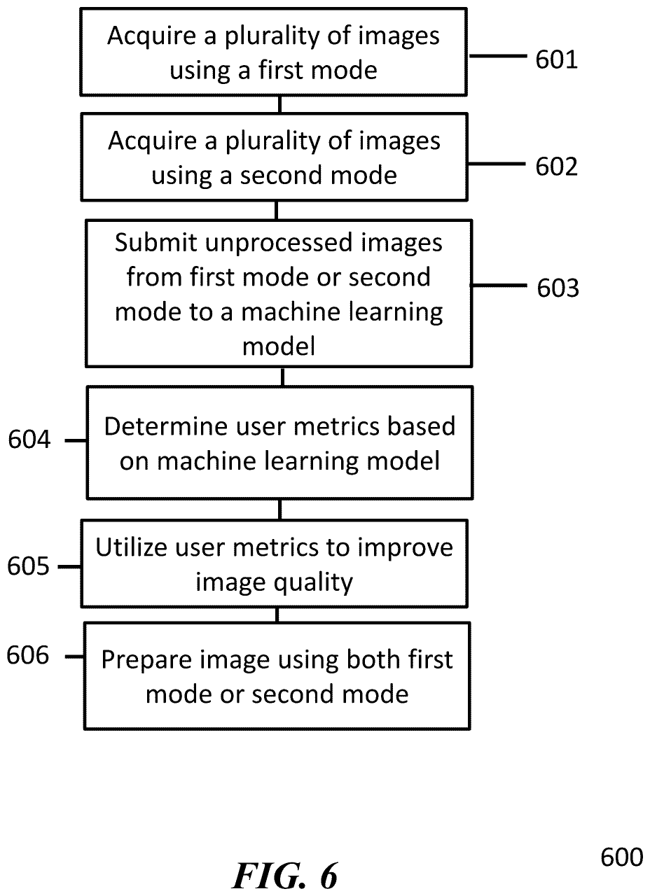

The workflow as described herein 600 is illustrated in FIG. 6. FIG. 6 comprises: acquiring a plurality of first mode images, for example, using B-mode to capture anatomical structures 601 and acquiring a plurality of images using second mode. The plurality of images captured using second mode, for example, use Color Doppler mode to capture blow flow movement in the anatomical structure. The second mode can use Zoom mode to capture high depth details of the anatomical structures 602. The plurality of images in both first mode and second may be captured, for example, using an imaging probe of an ultrasound imaging system (e.g., using a transducer of a two-dimensional ultrasound imaging system such as a linear probe, a curvilinear probe, or a phased array probe). The images may be unprocessed, wherein the plurality of images have not been processed for human readability. The unprocessed images will be directed to the ML model 603. The ML model may be used to determine user metrics 604. The user metrics may be used to enhance the raw image quality. The user metrics may provide a contemporaneous image quality score and/or a guided movement. The user metrics may be continually updated according to new processing data. The contemporaneous image quality score may provide a metric to quantify the image quality. The contemporaneous image quality score may be used to direct the guided movement. The contemporaneous image quality score may be used to further adjust the guided movement. The user metrics may be continually updated according to new processing data. The guided movements may direct the ML model to enhance the unprocessed images (e.g., reducing pixelation, speckle, clutter, or noise; and improving clarity and contrast) in order to improve image quality 605. The user metrics may be used to prepare the image 606. The workflow described herein enables the user to prepare a higher quality image as the aspects are processed only after submission and enhancement by the ML model. The workflow described in FIG. 6 ensures a holistic analysis of all the aspects of the image as opposed to an individual analysis of each aspect.

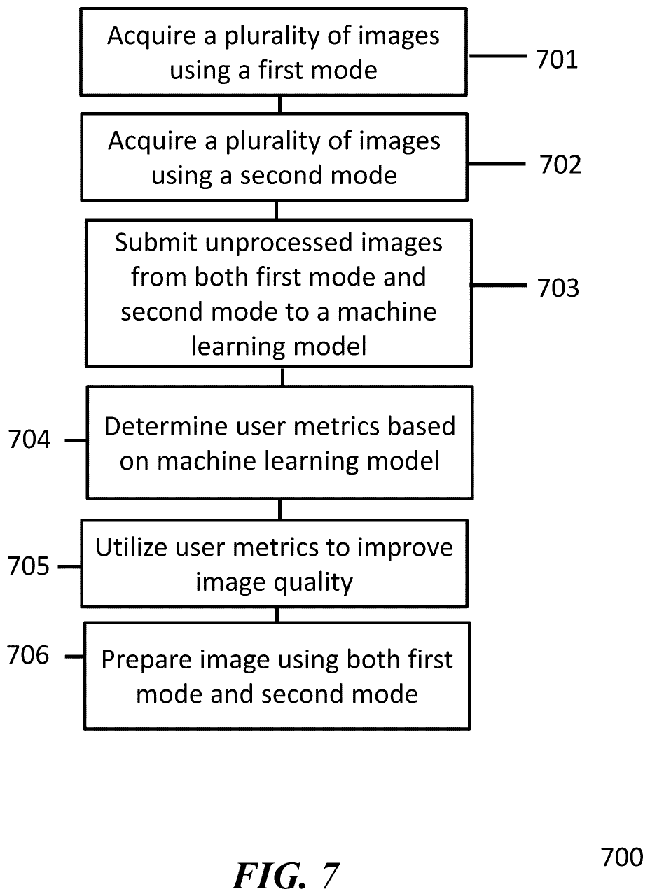

The example workflow 700 illustrated in FIG. 7 comprises: acquiring a plurality of first mode images, for example, using B-mode to capture anatomical structures 701 and acquiring a plurality of images using second mode. The plurality of images captured using second mode may, for example, use Color Doppler mode to capture blow flow movement in the anatomical structures. Conversely, second mode may use Zoom mode to capture high depth details of the anatomical structures 702. The plurality of images may be unprocessed. The plurality of unprocessed images may be captured, for example, using an imaging probe of an ultrasound imaging system (e.g., using a transducer of a two-dimensional ultrasound imaging system such as a linear probe, a curvilinear probe, or a phased array probe). The plurality of unprocessed images may not be altered for human readability. The plurality of unprocessed images can be submitted to a ML model 703. The ML model may be used to determine user metrics 704. The user metrics may be used to enhance raw image quality 705. The user metrics may provide a contemporaneous image quality score and/or a guided movement. The user metrics may be continually updated according to new processing data. The plurality of unprocessed images may be enhanced (e.g., reducing pixelation, clutter, speckle, or noise; and improving clarity and contrast) to improve image quality. The user metrics may be used to prepare the image according to both first mode and second mode 706.

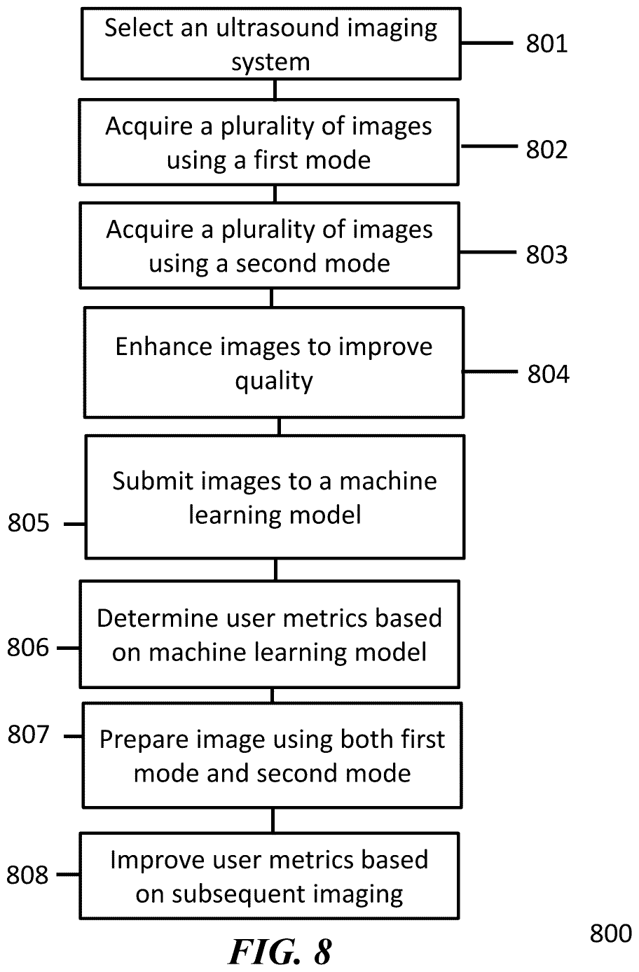

The example workflow 800, as illustrated in FIG. 8 comprises: selecting an ultrasound imaging system 801, acquiring a plurality of first mode images, for example, using B-mode to capture anatomical structures 802 and acquiring a plurality of images using second mode. The second mode may, for example, use Color Doppler mode to capture blow flow movement in the anatomical structures. The second mode can conversely use Zoom mode to capture high depth details of the anatomical structures 803. The plurality of images may be captured, for example, using an imaging probe of an ultrasound imaging system (e.g., using a transducer of a two-dimensional ultrasound imaging system such as a linear probe, a curvilinear probe, or a phased array probe). The plurality of images are processed, wherein the processed images may have been altered for human readability 804. The processed images may further be submitted to a ML model 805. The ML model may be used to determine user metrics 806. The user metrics may comprise a contemporaneous image quality score and/or a guided movement. The user metrics may be used to enhance the images to improve the image quality (e.g., reducing pixelation, speckle, clutter, or noise; and improving clarity and contrast). The user metrics may improve aspects of the image quality including but not limited to: clarity, contrast, threshold quality, and target view. The user metrics may be used to prepare the image 807. The user metrics may be continually updated according to new processing data 808. The contemporaneous image quality score may be accordingly used to direct the guided movement. Both the contemporaneous image quality score and the guided movement may be updated in view of new data submitted to the ML model.

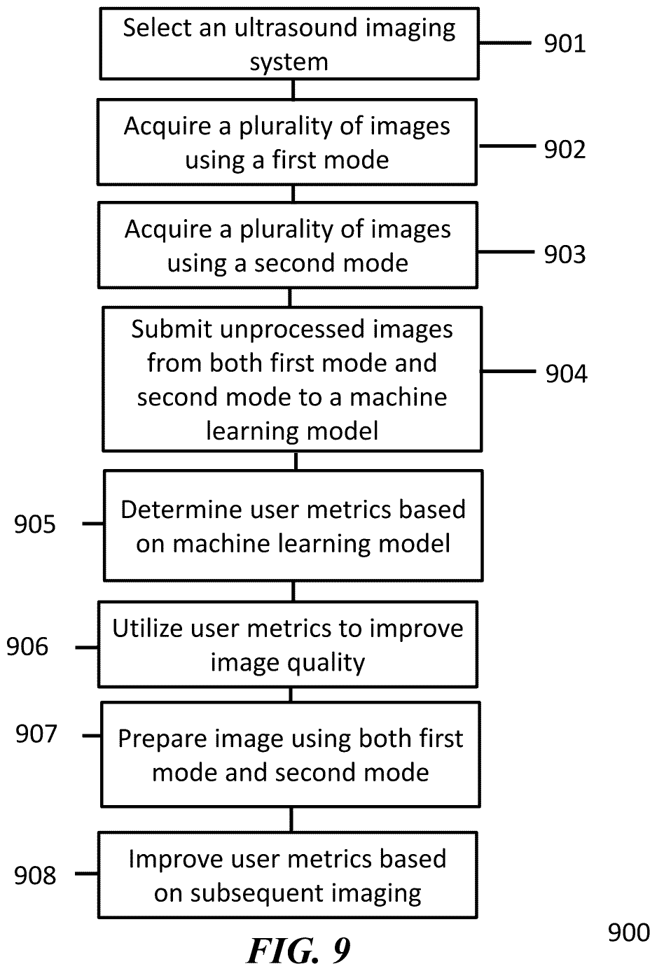

The example workflow 900 illustrated in FIG. 9 comprises: selecting an ultrasound imaging system 901, acquiring a plurality of images using first mode 902, for example, using B-mode to capture anatomical structures and acquiring a plurality of images using second mode. In some embodiments, second mode may, for example, using Color Doppler mode to capture blow flow movement in the anatomical structures. The second mode can use Zoom mode to capture high depth details of the anatomical structures 903. The plurality of images may be captured using an imaging probe of an ultrasound imaging system. The plurality of images may be unprocessed 904. The unprocessed images may further, for example, be submitted to a ML model 905. The ML model may be used to establish user metrics 906. The user metrics may provide a contemporaneous image quality score and/or a guided movement. The contemporaneous image quality score may further be used to direct the guided movement. The user metrics may be used to prepare the image 907. The user metrics may be used to enhance the image quality (e.g., reducing pixelation, clutter, speckle, or noise and improving clarity and contrast). The user metrics may be continually improved 908. The contemporaneous image quality score likewise may be updated according to new image data.

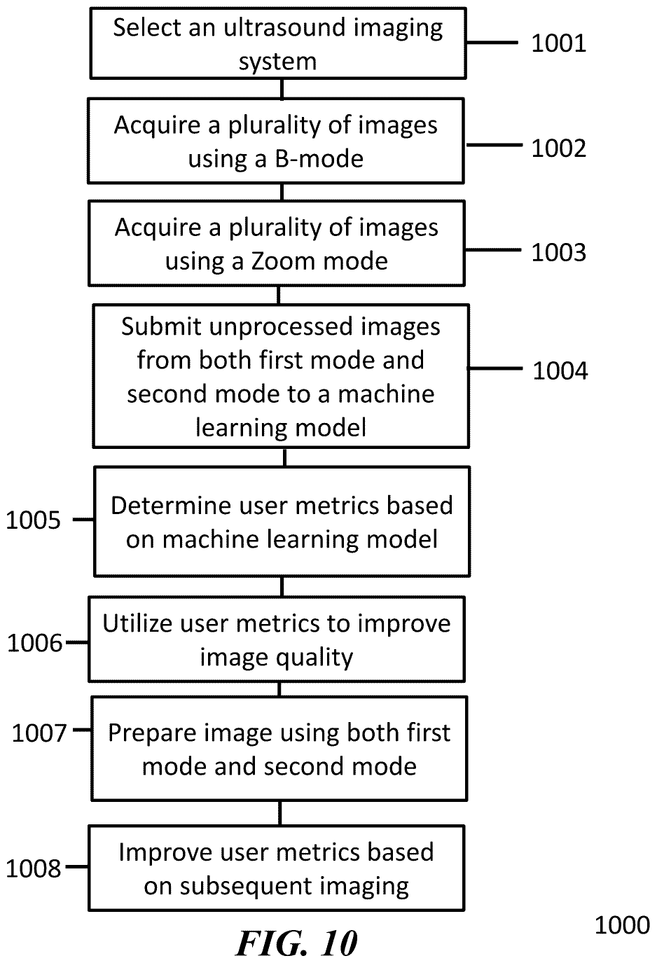

The example workflow 1000 illustrated in FIG. 10 comprises: selecting an ultrasound imaging system 1001, acquiring a plurality of images using B-mode to capture anatomical structures 1002 and acquiring a plurality of images using zoom mode to capture high depth details of the anatomical structures 1003. The plurality of images may be captured using an imaging probe of an ultrasound imaging system. The plurality of images may be unprocessed. In some embodiments, the unprocessed images are not altered for human readability. The unprocessed images may further, for example, be submitted to a ML model 1004. The machine learning model may be used to establish user metrics 1005. The user metrics may be used to improve image quality. The user metrics may provide a contemporaneous image quality score and/or a guided movement. The contemporaneous image quality score may further be used to direct the guided movements. The guided movements may improve the quality score by enhancing the raw plurality of images. The enhancements may include but not limited to: quality reducing pixelation, clutter, speckle, or noise; and improving clarity and contrast 1006. The user metrics may be used to prepare the image according to both first mode and second mode 1007. The user metrics may be continually improved. The user metrics may provide improved user metrics based on subsequent imaging 1008.

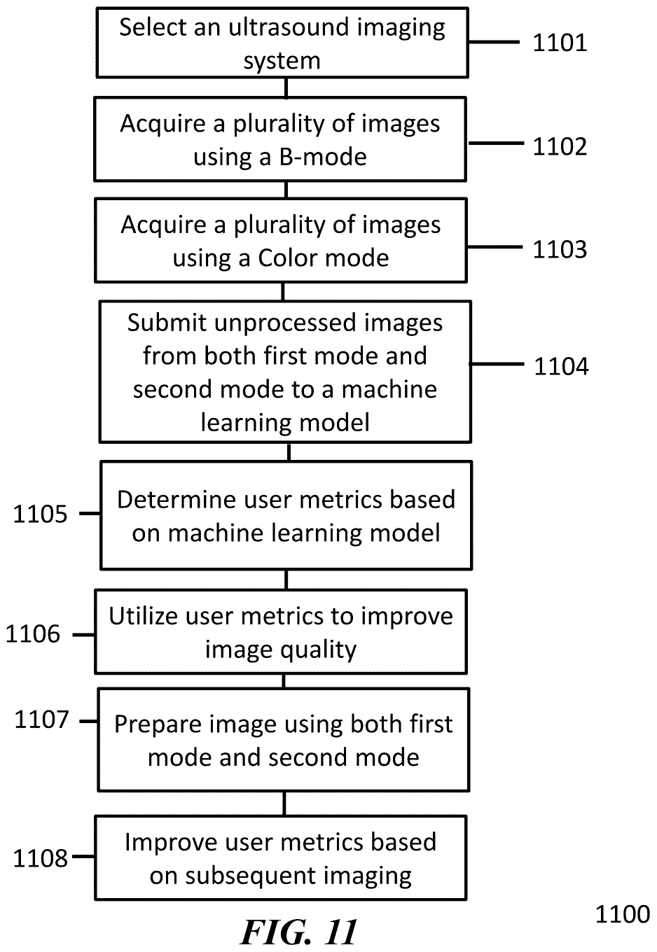

The example workflow 1100 illustrated in FIG. 11 comprises: selecting an ultrasound imaging system 1101, acquiring a plurality of images using B-mode to capture anatomical structures 1102 and acquiring a plurality of images using Color Doppler mode mode to capture high depth details of the blood flow movement in the anatomical structure 1103. The plurality of images may be captured using an imaging probe of an ultrasound imaging system. The plurality of images may be unprocessed. In some embodiments, the plurality of unprocessed images are not altered for human readability. The plurality of unprocessed images may further be submitted to a ML model 1104. The ML model may establish user metrics 1105. The user metrics may be determined according to both first mode and second mode data. The user metrics may be used to improve image quality. The user metrics may provide a contemporaneous image quality score and/or a guided movement. The contemporaneous image quality score may further direct the guided movements. The guided movements may improve the quality score by enhancing the plurality of raw images. The guided movements may improve aspects of the image including but not limited to: reducing pixelation, clutter, speckle, or noise; and improving clarity and contrast 1106. The user metrics may be used to prepare the image according to both first mode and second mode processes 1107. The user metrics may be continually improved. The user metrics may provide improved user metrics based on subsequent imaging 1108.

### Example Images

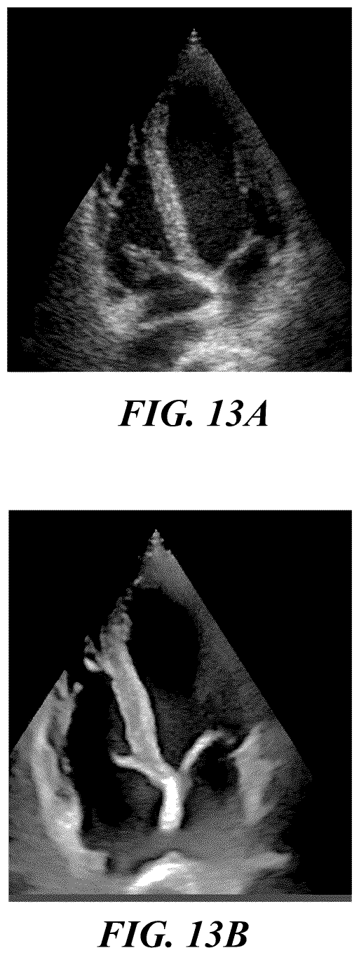

FIGS. 13A-B provide an example of downstream cardiac ultrasound images. The images depict the challenges with highly processed issues being submitted to a machine learning system. In FIG. 13A, the processed image has clutter, speckle, and noise, which presents a distraction to the user. FIG. 13B is a processed image with low clarity and contrast. In both FIGS. 13A and 13B, the processed images can present a challenge to the machine learning system because the enhancements can distract the machine learning system from properly identifying the targets in the image.

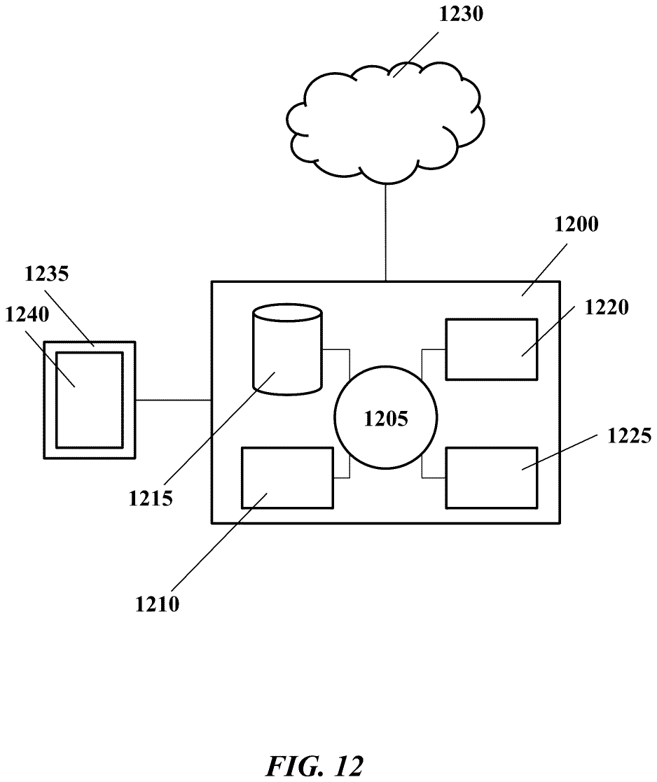

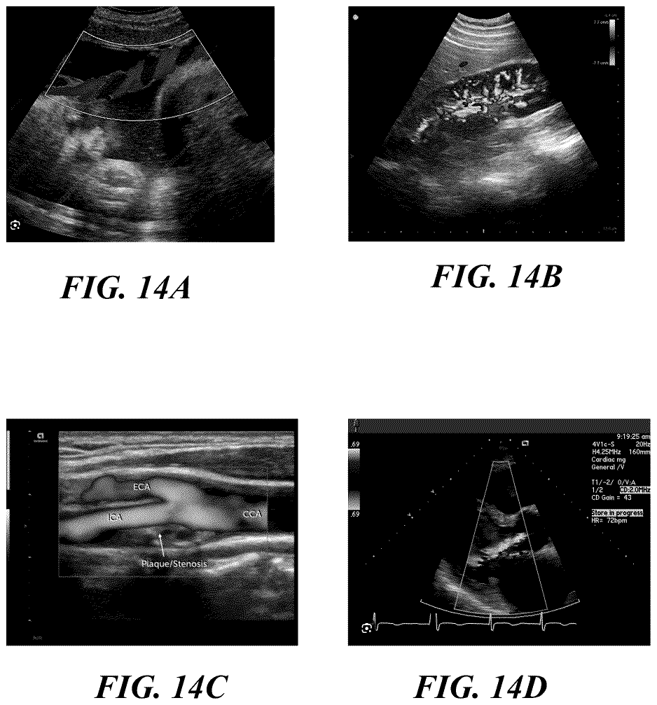

FIGS. 14A-D provide an example of the images captured using the dual-mode guided ultrasound system, wherein the first mode is B-mode and the second mode is Color Doppler Mode. FIG. 14A depicts the umbilical artery, FIG. 14B depicts the kidney blood flow, FIG. 14C depicts the carotid artery, and FIG. 12D depicts aortic valve regurgitation.

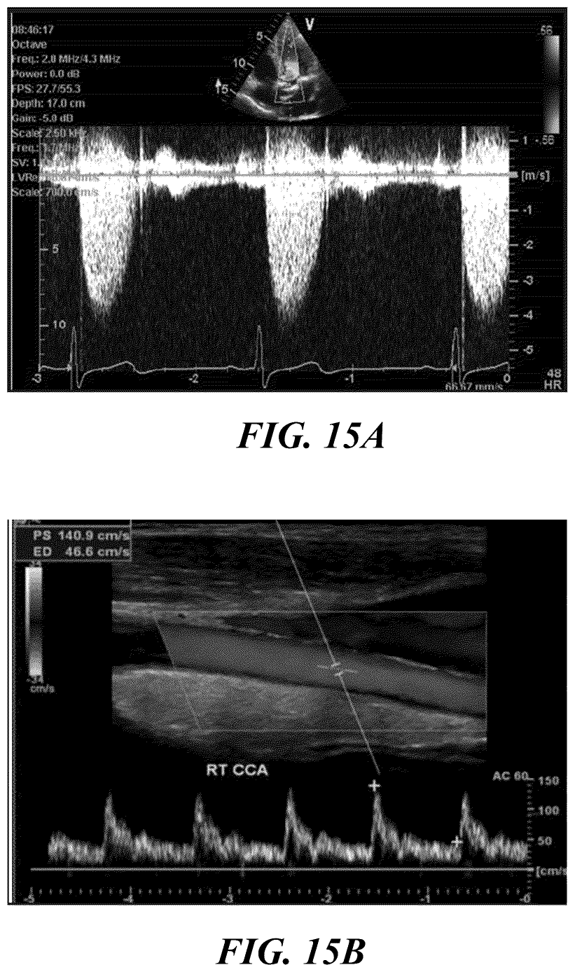

FIGS. 15A-B depict a heart using the Spectral Doppler mode. In some embodiments, Spectral Doppler mode is a form of Doppler blood flow mapping. In some embodiments, Spectral Doppler mode displays a spectral histogram of the blood flow velocities. In some embodiments, Spectral Doppler mode is performed along with B-mode. In some embodiments, Spectral Doppler mode is performed along with B-mode and/or along with B-mode and Color mode. In some embodiments, FIGS. 15A-B depicts an overlay of Spectral Doppler mode, Color mode, and B-mode imaging of the human body. In some embodiments, FIG. 15A depicts an overlay of Spectral Doppler mode, Color mode, and B-mode imaging of the heart. In some embodiments, FIG. 15B depicts the overlay of Spectral Doppler mode, Color mode, and B-mode imaging of the carotid artery.

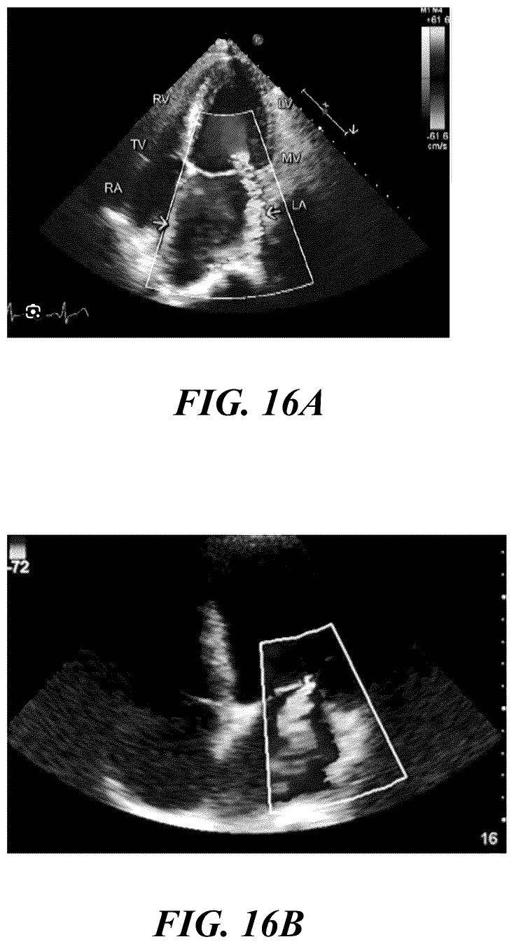

FIG. 16A depict the optimal color blood flow, wherein the quality of the 2D image is reduced in order to enhance the blood flow movement. FIG. 16A depicts the apical four chamber view of mitral valve regurgitation. The arrow points to the mitral regurgitation where blood is flowing backwards into the left atrium during systole when the closed mitral valve is leaking. FIG. 16B also demonstrates mitral regurgitation. The left ventricular walls are poorly visualized and the apex of the heart is almost completely missing or not displayed. The tricuspid valve is not seen and the right atrium and right ventricle are poorly delineated. While the blood flow is captured well, the anatomical features are not detectable demonstrating the limitations in using a single mode to capture different processes. The methods and systems described herein present two separate models at the same time or create a hybrid that analyzes both types of data.

### Diagnostic Image Quality

A particular challenge in ultrasound medical imaging is accurately determining what probe pose or movement may provide a clinical or diagnostic quality image. As used herein, an image quality (e.g., diagnostic quality or clinical quality) may be used to refer to one or more aspects of the quality of an image. In some aspects, image quality is in reference to an image that can be viewed by a trained expert or a machine learning tool in a way that anatomy is identified and a diagnostic interpretation can be made. In some aspects, image quality is in reference to an image in which the targets are displayed in a clear and well-defined manner, for example, where extraneous noise or clutter is minimal, the grayscale display shows subtle variations of tissue type and texture, frame rates are high, providing accurate depiction of tissue, aeration or lack thereof, or blood flow movement, borders between tissue types or blood flow and vessel or other structures are well resolved, ultrasound artifacts such as grating and side lobes are minimized, acoustic noise is absent, places to make measurements in the image are obvious and distinct, or any combination thereof depending on the nature of the ultrasound exam. In some aspects, image quality is in reference to an image that contains the necessary anatomical targets to represent a standard diagnostic view. In some aspects, image quality is in reference to an image in which a diseased condition, abnormality, or pathology is well visualized. For example, medical images may be labeled by healthcare professionals according to whether they are considered to have a well visualized diseased condition, abnormality, or pathology, and then used to train a machine learning algorithm to differentiate between images based on image quality. In some aspects, image quality means that some combination of these aforementioned characteristics is present.

In some aspects, the plurality of images captures are unprocessed prior to submitting to the machine learning systems. Unprocessed images can also be described as raw images. In some embodiments, the images not changed, edited, or manipulated. The images are sent directly to the machine learning system so as to develop the user metrics.

Accordingly, disclosed herein are platforms, systems, and methods comprising one or more algorithms for evaluating ultrasound images to provide user metrics. In some aspects, the user metrics are provided in real time. The user metrics may further be updated in real-time in response to new data.

In some embodiments, the user metrics comprise a contemporaneous image quality score and/or guided movements. The contemporaneous image quality score may provide data regarding image quality. In some embodiments, the score provides a threshold quality. The user may utilize the contemporaneous image quality score to evaluate the accuracy of the depicted image. The user may utilize the score to determine whether additional images are required. In some embodiments, replacement images are required.

The user metrics may be used to improve the image quality. In some embodiments, the guided movement may alter the images. In some embodiments, the guided movement may be updated according to the contemporaneous image quality score.

In some embodiments, the user metrics include one or more guided movements. The guided movements may include changing settings, moving the probe, and/or moving the patient or subject position. In some embodiments, the user metrics may direct the guided movement to enhance the image quality (e.g., improve clarity and contrast; and reduce pixelation, speckle, clutter, and noise). In some embodiments, the improvement in image quality improves the visualization of a target structural feature or a target organ of a subject. In some embodiments, the user metrics can be continually updated in response to new data.

### Machine Learning Algorithms

Disclosed herein are platforms, systems, and methods that provide ultrasound image classification using machine learning algorithm(s). The ML and AI system described herein can be used to calculate user metrics. In some embodiments, the user metrics can provide a contemporaneous image quality score and/or guided movements.

In particular, in some aspects, the machine learning algorithms include deep learning neural networks configured for evaluating ultrasound images. The algorithms can include one or more of a positioning algorithm, a scoring algorithm, a probe guidance algorithm, and an intrinsic image quality algorithm. The positioning algorithm can include one or more neural networks that estimate probe positioning relative to an ideal anatomical view or perspective and/or a distance or deviation of a current probe position from an ideal probe position. The intrinsic image quality algorithm may determine that intrinsic image quality is below a threshold based in part on a determination by a positioning algorithm that one or more images have been acquired at a probe position expected to obtain a clinical quality image.

The development of each machine learning algorithm spans three phases: (1) dataset creation and curation, (2) algorithm training, and (3) adapting design elements necessary for product performance and useability. The dataset used for training the algorithm can be generated by obtaining the user metrics from a plurality of images captured under first and/or second mode. Each algorithm then can be modified using data collected from the plurality of images, which can include one or more different target organs and/or one or more different views of a given target organ.

A machine learning model can comprise a supervised, semi-supervised, unsupervised, or self-supervised machine learning model. In some cases, the one or more ML approaches perform classification or clustering of the MS data. In some examples, the machine learning approach comprises a classical machine learning method, such as, but not limited to, support vector machine (SVM) (e.g., one-class SVM, linear or radial kernels, etc.), K-nearest neighbor (KNN), isolation forest, random forest, logistic regression, AdaBoost classifier, extra trees classifier, extreme gradient boosting, gaussian process classifier, gradient boosting classifier, light gradient boosting, linear discriminant analysis, naïve Bayes, quadratic discriminant analysis, ridge classifier, or any combination thereof. In some examples, the machine learning approach comprises a deep leaning method (e.g., deep neural network (DNN)), such as, but not limited to a fully-connected network, convolutional neural network (CNN) (e.g., one-class CNN), recurrent neural network (RNN), transformer, graph neural network (GNN), convolutional graph neural network (CGNN), multi-level perceptron (MLP), or any combination thereof.

In some aspects, a classical ML method comprises one or more algorithms that learns from existing observations (i.e., known features) to predict outputs. In some aspects, the one or more algorithms perform clustering of data. In some examples, the classical ML algorithms for clustering comprise K-means clustering, mean-shift clustering, density-based spatial clustering of applications with noise (DBSCAN), expectation-maximization (EM) clustering (e.g., using Gaussian mixture models (GMM)), agglomerative hierarchical clustering, or any combination thereof. In some aspects, the one or more algorithms perform classification of data. In some examples, the classical ML algorithms for classification comprise logistic regression, naïve Bayes, KNN, random forest, isolation forest, decision trees, gradient boosting, support vector machine (SVM), or any combination thereof. In some examples, the SVM comprises a one-class SMV or a multi-class SVM.

In some aspects, the deep learning method comprises one or more algorithms that learns by extracting new features to predict outputs. In some aspects, the deep learning method comprises one or more layers. In some aspects, the deep learning method comprises a neural network (e.g., DNN comprising more than one layer). Neural networks generally comprise connected nodes in a network, which can perform functions, such as transforming or translating input data. In some aspects, the output from a given node is passed on as input to another node. The nodes in the network generally comprise input units in an input layer, hidden units in one or more hidden layers, output units in an output layer, or a combination thereof. In some aspects, an input node is connected to one or more hidden units. In some aspects, one or more hidden units is connected to an output unit. The nodes can generally take in input through the input units and generate an output from the output units using an activation function. In some aspects, the input or output comprises a tensor, a matrix, a vector, an array, or a scalar. In some aspects, the activation function is a Rectified Linear Unit (ReLU) activation function, a sigmoid activation function, a hyperbolic tangent activation function, or a Softmax activation function.

The connections between nodes further comprise weights for adjusting input data to a given node (i.e., to activate input data or deactivate input data). In some aspects, the weights are learned by the neural network. In some aspects, the neural network is trained to learn weights using gradient-based optimizations. In some aspects, the gradient-based optimization comprises one or more loss functions. In some aspects, the gradient-based optimization is gradient descent, conjugate gradient descent, stochastic gradient descent, or any variation thereof (e.g., adaptive moment estimation (Adam)). In some further aspects, the gradient in the gradient-based optimization is computed using backpropagation. In some aspects, the nodes are organized into graphs to generate a network (e.g., graph neural networks). In some aspects, the nodes are organized into one or more layers to generate a network (e.g., feed forward neural networks, convolutional neural networks (CNNs), recurrent neural networks (RNNs), etc.). In some aspects, the CNN comprises a one-class CNN or a multi-class CNN.

In some aspects, the neural network comprises one or more recurrent layers. In some aspects, the one or more recurrent layers are one or more long short-term memory (LSTM) layers or gated recurrent units (GRUs). In some aspects, the one or more recurrent layers perform sequential data classification and clustering in which the data ordering is considered (e.g., time series data). In such aspects, future predictions are made by the one or more recurrent layers according to the sequence of past events. In some aspects, the recurrent layer retains or “remembers” important information, while selectively “forgets” what is not essential to the classification.

In some aspects, the neural network comprise one or more convolutional layers. In some aspects, the input and the output are a tensor representing variables or attributes in a data set (e.g., features), which may be referred to as a feature map (or activation map). In such aspects, the one or more convolutional layers are referred to as a feature extraction phase. In some aspects, the convolutions are one dimensional (1D) convolutions, two dimensional (2D) convolutions, three dimensional (3D) convolutions, or any combination thereof. In further aspects, the convolutions are 1D transpose convolutions, 2D transpose convolutions, 3D transpose convolutions, or any combination thereof.

The layers in a neural network can further comprise one or more pooling layers before or after a convolutional layer. In some aspects, the one or more pooling layers reduces the dimensionality of a feature map using filters that summarize regions of a matrix. In some aspects, this down samples the number of outputs, and thus reduces the parameters and computational resources needed for the neural network. In some aspects, the one or more pooling layers comprises max pooling, min pooling, average pooling, global pooling, norm pooling, or a combination thereof. In some aspects, max pooling reduces the dimensionality of the data by taking only the maximums values in the region of the matrix. In some aspects, this helps capture the most significant one or more features. In some aspects, the one or more pooling layers is one dimensional (1D), two dimensional (2D), three dimensional (3D), or any combination thereof.

The neural network can further comprise of one or more flattening layers, which can flatten the input to be passed on to the next layer. In some aspects, a input (e.g., feature map) is flattened by reducing the input to a one-dimensional array. In some aspects, the flattened inputs can be used to output a classification of an object. In some aspects, the classification comprises a binary classification or multi-class classification of visual data (e.g., images, videos, etc.) or non-visual data (e.g., measurements, audio, text, etc.). In some aspects, the classification comprises binary classification of an image (e.g., contrast needed or contrast not needed). In some aspects, the classification comprises multi-class classification of a text (e.g., identifying hand-written digits)). In some aspects, the classification comprises binary classification of a measurement. In some examples, the binary classification of a measurement comprises a classification of a system's performance using the physical measurements described herein (e.g., normal or abnormal, normal or anormal).

The neural networks can further comprise of one or more dropout layers. In some aspects, the dropout layers are used during training of the neural network (e.g., to perform binary or multi-class classifications). In some aspects, the one or more dropout layers randomly set some weights as 0 (e.g., about 10%, 20%, 30%, 40%, 50%, 60%, 70%, 80% of weights). In some aspects, the setting some weights as 0 also sets the corresponding elements in the feature map as 0. In some aspects, the one or more dropout layers can be used to avoid the neural network from overfitting.

The neural network can further comprise one or more dense layers, which comprises a fully connected network. In some aspects, information is passed through a fully connected network to generate a predicted classification of an object. In some aspects, the error associated with the predicted classification of the object is also calculated. In some aspects, the error is backpropagated to improve the prediction. In some aspects, the one or more dense layers comprises a Softmax activation function. In some aspects, the Softmax activation function converts a vector of numbers to a vector of probabilities. In some aspects, these probabilities are subsequently used in classifications, such as classifications of one or a plurality of image features comprised in one or a plurality of ultrasound images of a lung of a subject.

### Computer Systems

The present disclosure provides computer systems that are programmed to implement methods of the disclosure. FIG. 12 shows a computer system 1200 that is programmed or otherwise configured to assess whether an ultrasound enhancing agent is expected to improve image quality according to any of the methods described herein. The computer system 1200 can regulate various aspects of the present disclosure. The computer system 1200 can be an electronic device of a user or a computer system that is remotely located with respect to the electronic device. The electronic device can be a mobile electronic device.

The computer system 1200 includes a central processing unit (CPU, also “processor” and “computer processor” herein) 1205, which can be a single core or multi core processor, or a plurality of processors for parallel processing. The computer system 1200 also includes memory or memory location 1210 (e.g., random-access memory, read-only memory, flash memory), electronic storage unit 1215 (e.g., hard disk), communication interface 1220 (e.g., network adapter) for communicating with one or more other systems, and peripheral devices 1225, such as cache, other memory, data storage and/or electronic display adapters. The memory 1210, storage unit 1215, interface 1220 and peripheral devices 1225 are in communication with the CPU 1205 through a communication bus (solid lines), such as a motherboard. The storage unit 1215 can be a data storage unit (or data repository) for storing data. The computer system 1200 can be operatively coupled to a computer network (“network”) 1230 with the aid of the communication interface 1220. The network 1230 can be the Internet, an internet and/or extranet, or an intranet and/or extranet that is in communication with the Internet. The network 1230 in some cases is a telecommunication and/or data network. The network 1230 can include one or more computer servers, which can enable distributed computing, such as cloud computing. The network 1230, in some cases with the aid of the computer system 1200, can implement a peer-to-peer network, which may enable devices coupled to the computer system 1200 to behave as a client or a server.

The CPU 1205 can execute a sequence of machine-readable instructions, which can be embodied in a program or software. The instructions may be stored in a memory location, such as the memory 1210. The instructions can be directed to the CPU 1205, which can subsequently program or otherwise configure the CPU 1205 to implement methods of the present disclosure. Examples of operations performed by the CPU 1205 can include fetch, decode, execute, and writeback.

The CPU 1205 can be part of a circuit, such as an integrated circuit. One or more other components of the system 1200 can be included in the circuit. In some cases, the circuit is an application specific integrated circuit (ASIC).

The storage unit 1215 can store files, such as drivers, libraries, and saved programs. The storage unit 1215 can store user data, e.g., user preferences and user programs. The computer system 1200 in some cases can include one or more additional data storage units that are external to the computer system 1200, such as located on a remote server that is in communication with the computer system 1200 through an intranet or the Internet.

The computer system 1200 can communicate with one or more remote computer systems through the network 1230. For instance, the computer system 1200 can communicate with a remote computer system of a user (e.g., a professional sonographer or an untrained technician). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1200 via the network 1230.

Methods as described herein can be implemented by way of machine (e.g., computer processor) executable code stored on an electronic storage location of the computer system 1200, such as, for example, on the memory 1210 or electronic storage unit 1215. The machine executable or machine readable code can be provided in the form of software. During use, the code can be executed by the processor 1205. In some cases, the code can be retrieved from the storage unit 1215 and stored on the memory 1210 for ready access by the processor 1005. In some situations, the electronic storage unit 1215 can be precluded, and machine-executable instructions are stored on memory 1210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1200, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1200 can include or be in communication with an electronic display 1035 that comprises a user interface (UI) 1240 for providing, for example, providing a user with an indication of whether or not an ultrasound enhancing agent is needed to improve an intrinsic image quality. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1205. The algorithm can, for example, be configured to perform any of the methods described herein.

### Ultrasound Annotations

In some embodiments, the unprocessed images are not processed according to user metrics. In some embodiments, the unprocessed images are not processed according to a guided movement. In some embodiments, the unprocessed images are not processed according to a contemporaneous image quality score. In some embodiments, the unprocessed images are not processed according to first mode. In some embodiments, the unprocessed images are not processed according to B-mode. In some embodiments, the unprocessed images are not processed according to Spectral Doppler mode. In some embodiments, the unprocessed images are not processed according to second mode. In some embodiments, the unprocessed images are not processed according to Color Doppler mode. In some embodiments, the unprocessed images are not processed according to Zoom mode.

In some embodiments, the unprocessed images are not processed in a displayable format. In some embodiments, the unprocessed images are not processed for human readability. In some embodiments, the unprocessed images are not processed for human readability of anatomical features. The anatomical features may include but are not limited to: the pancreas, liver, gall bladder, kidney, spleen, heart, and/or the reproductive system. The unprocessed images may not be processed for human readability of a specific target on the anatomical feature. In some embodiments, the specific targets are on the left or right side of the anatomical feature. In some embodiments, the specific targets are up or down on the anatomical feature.

In some embodiments, the unprocessed images are not processed using a transducer. The transducer may be a convex/curvilinear transducer, a linear transducer, or a phased/sector transducer. In some embodiments, the unprocessed images are not processed for 2D processing. In some embodiments, the unprocessed images are not processed for Color Doppler processing. In some embodiments, the unprocessed images are not processed for Spectral Doppler processing. In some embodiments, the unprocessed images are not processed for Doppler blood flow mapping. In some embodiments, the unprocessed images are not processed for a Spectral histogram. In some embodiments, the Spectral histogram displays the blood flow velocities. In some embodiments, the unprocessed images do not undergo scan conversion. In some embodiments, the unprocessed images do not undergo image enhancement processing.

In some embodiments, the unprocessed images do not produce a user readable structural image. In some embodiments, the unprocessed images are not processed for human readable structural features. The structural features may include but are not limited to: the anatomical boundaries, shapes, and relative positions of different tissues and organs. The unprocessed images may not be processed for ultrasound readouts. The ultrasound readouts may be used to indicate the density of tissue. In some embodiments, the black areas indicate fluid. In some embodiments, the brightest white indicate bone. In some embodiments, the unprocessed images are not processed for the movement of blood flow in the anatomical structures.

In some embodiments, the unprocessed images are not processed for a target current. In some embodiments, the unprocessed images are not processed for a threshold quality. In some embodiments, the unprocessed images are not processed for a target view. In some embodiments, the unprocessed images are not processed to improve image quality. In some embodiments, the unprocessed images are not enhanced to improve raw image quality.

In some embodiments, the unprocessed images are not processed to increase clarity and/or contrast. In some embodiments, the unprocessed images are not processed to retain, retune, and/or retest. In some embodiments, the unprocessed images are not processed for an internal scan. In some embodiments, the unprocessed images are not processed for an external scan. In some embodiments, the unprocessed images are not processed for a diagnostic procedure. In some embodiments, the unprocessed images are not processed in real-time. In some embodiments, the unprocessed images are not processed under a ML system. In some embodiments, the unprocessed images are not processed using AI. In some embodiments, the unprocessed images are not processed to magnify depth, angle, and/or line density. In some embodiments, the unprocessed images are not processed for an electrocardiogram (i.e., EKG), abdominal scan, obstetric ultrasound, Doppler ultrasound, transabdominal ultrasound, transrectal ultrasound, thyroid scan, transvaginal scan, testicular scan, carotid ultrasound, venosus ultrasound, 3D ultrasound, and/or a 4D ultrasound.

### EXAMPLES

### Example 1: Machine Learning Models for Guidance of Imaging Procedures Utilizing Dual-Mode Guides

Described herein are dual-mode guided imaging system or process having a plurality of unprocessed images. In some instances, the plurality of images are unprocessed prior to submission to the machine learning (ML) model. In other imaging systems described herein, images are altered for human readability, such as instructed by a human, prior to submission to a ML model. In some instances, images altered for human readability before submission to the ML model prioritizes one aspect of the images, such as misleading the system to analyze the plurality of images according to the prioritized aspect, leading to overall lower image quality. In some embodiments, the dual-mode guided imaging system or process described herein does not alter the images for human readability, such as until after enhancements from the ML model. In some instances, the dual-mode ultrasound imaging system or process described herein provides improved image quality because the images do not undergo alterations, such as prior to submission to the ML model. The dual-mode ultrasound imaging system or process described herein, in some instances, provides improved image quality associated with reduced pixelation, clutter, and noise and increased clarity and contrast.

The imaging process for the dual-mode ultrasound imaging system or process described herein encompasses a front end and a back end. The front end includes the transmit circuit control, transducer, beam former, and RF processing. The back end includes the first and second mode, scan conversion, Machine Learning (ML) and Artificial Intelligence (AI) processing, image enhancement processing, and image display and storage. The dual-mode ultrasound imaging system or process comprises a computer and a probe. The probe may be a wireless probe. The probe can integrate the front end processes including the transmit circuit control, transducer, and the beamforming. The wireless probe can perform all the functions related to transmitting, receiving, and beamforming in the probe housing. Scan conversion may also be performed in the probe. If scan conversion is software-based, it may also be done on a tablet or phone which the probe is connected to.

The dual-mode ultrasound imaging system or process described herein may be selected to capture a plurality of images (see FIG. 9). The dual-mode ultrasound imaging system or process can acquire a plurality of images using a first mode 902. In some cases, the first mode is B-mode. The dual-mode ultrasound imaging system or process can further acquire a plurality of images using a second mode 903. In some cases, the second mode is Zoom mode (see FIG. 10). In some cases, the second is Color Doppler mode (see FIG. 11). In some instances, the unprocessed images from both the first mode and second mode are submitted to a ML model 904. In some instances, the ML model further determines user metrics 905. In some instances, the user metrics can be used to enhance the images by reducing pixelation, clutter, or noise and/or improving clarity and contrast. In some instances, the ML model described herein provides the user metrics. The metrics can include a contemporaneous image quality score and/or a guided movement. The contemporaneous image quality score can provide a metric to the user to direct the guided movement. The guided movement can improve the image quality by, for example, increasing clarity or contrast 906. The final image can be prepared according to user metrics determined from both the first mode and second mode 907.

The user metrics can update in response to additional imaging. The user metrics may improve in response to subsequent imaging 908.

The user metrics may provide a quality meter in the user interface of the system. Upon acquisition of a first image, a user reads the quality meter and may respond by moving the probe in a plurality of directions. The user may rotate, tilt, and/or slide the ultrasound transducer probe. The quality meter may be updated with each subsequently acquired image. The user may further continue moving the probe in a direction, wherein the probe further rotates, tilts, and/or slides. The user may choose to continue moving the probe until a threshold clinical quality is obtained and/or the quality of a subsequently acquired image is improved. The guided movements may be detected and automatically saved by the ML system.

What is claimed is:

1. An ultrasound imaging system configured for conducting a dual-mode guided ultrasound
imaging procedure, the system comprising:
an ultrasound imaging probe;
a computing system; and
a non-transitory computer-readable storage medium, storing instructions that, when executed by a processor of the computing system cause the ultrasound imaging system to:
obtain a first plurality of images comprising a first mode and a second mode, the first mode comprising a B-mode ultrasound imaging mode, and the second mode comprising a Doppler flow ultrasound imaging mode;
submit an unprocessed first mode image of the first plurality of images to a machine learning model to determine one or more user metrics comprising a contemporaneous image quality score and/or a guided movement expected to improve an image quality of a subsequently acquired second plurality of images compared to an image quality of the first plurality of images, based at least in part on the unprocessed first mode image of the first plurality of images; and
provided the one or more user metrics to a user of the ultrasound imaging system.

2. A method for conducting a dual-mode guided ultrasound imaging produce, the method comprising:
obtaining a first plurality of images from an ultrasound imaging system, the first plurality of images comprising a first mode and a second mode, the first mode comprising a B-mode ultrasound imaging mode, and the second mode comprising a Doppler flow ultrasound imaging mode;
submitting an unprocessed first mode image of the first plurality of images to a machine learning model to determine one or more user metrics comprising a contemporaneous image quality score and/or a guided movement expected to improve an image quality of a subsequently acquired second plurality of images compared to an image quality of the first plurality of images, based at least in part on the unprocessed first mode image of the first plurality of images; and
providing the one or more user metrics are provided to a user of the ultrasound imaging system.

3. The ultrasound imaging system of claim 1, wherein the first plurality of images comprises multiple, repetitive images.

4. The ultrasound imaging system of claim 1, wherein the dual-mode guided ultrasound imaging procedure identifies a target organ.

5. The ultrasound imaging system of claim 1, wherein the B-mode depicts an anatomical structure.

6. The ultrasound imaging system of claim 1, wherein the Doppler flow ultrasound imaging mode comprises providing a color map of blood flow together with a user readable structural image of the first mode.

7. The ultrasound imaging system of claim 1, wherein the Doppler flow ultrasound imaging mode comprises providing a spectral histogram of blood flow together with the user readable structural image of the first mode.

8. The ultrasound imaging system of claim 1, wherein the improvement in image quality improves the raw image quality.

9. The ultrasound imaging system of claim 8, wherein the improvement in image quality comprises an improvement in visualization of a target structural feature of the target organ of a subject.

10. The ultrasound imaging system of claim 1, further comprising:
(d) the first mode image of the second plurality of images;
(e) the unprocessed first mode image of (d) is submitted to a machine learning model to obtain one or more updated user metrics; and
(f) the updated user metrics are provided to the user.

11. The ultrasound imaging system of claim 10, further comprising repeating (d) and (e) in real-time.

12. The ultrasound imaging system of claim 11, wherein the user metrics are provided until a target view is reached.

13. The ultrasound imaging system of claim 12, wherein the target view is determined based at least in part on the diagnostic procedure.

14. The ultrasound imaging system of claim 13, wherein the user metrics are provided until a threshold quality is reached.

15. The ultrasound imaging system of claim 14, wherein the threshold quality depends upon a presence of a target structural feature of an organ, a clarity of structural image quality, Doppler image quality, or a combination thereof.

16. The ultrasound imaging system of claim 15, wherein blood flow takes place in the target organ.

17. The ultrasound imaging system of claim 1, wherein an unprocessed second mode data is used to determine the user metrics.

18. The ultrasound imaging system of claim 1, wherein the first mode is used to determine one or more aspects including current, threshold quality, or target view; and wherein the second mode is used to determine one or more aspects including current, threshold quality, or target view.

19. The ultrasound imaging system of claim 1, wherein the unprocessed first mode image comprises a plurality of raw images that have not been enhanced for human-readability and are not in a displayable format; and wherein the unprocessed second mode image comprises a plurality of raw images that have not been enhanced for human-readability and are not in a displayable format.

20. A computer system for conducting a dual-mode guided ultrasound imaging procedure, the computer system configured to:
receive a first plurality of images comprising a first mode and a second mode, the first mode comprising a B-mode ultrasound imaging mode, and the second mode comprising a Doppler flow ultrasound imaging mode;
submit an unprocessed first mode image of the first plurality of images to a machine learning model to determine one or more user metrics comprising a contemporaneous image quality score and/or a guided movement expected to improve an image quality of a subsequently acquired second plurality of images compared to an image quality of the first plurality of images, based at least in part on the unprocessed first mode image of the first plurality of images; and
provide the one or more user metrics to a user of the ultrasound imaging system.
